(19) United States
(12) Patent Application Publication
(10) Pub. No.: US 2022/0165400 A1
(43) Pub. Date: May 26, 2022

(54) "NOTIFYME" AUTONOMOUS REAL-TIME REMOTE NOTIFICATION OF INDIVIDUAL SCAN PROTOCOL STATUS AND MRI EXAM PROGRESS

(71) Applicant: KONINKLIJKE PHILIPS N.V., EINDHOVEN (NL)

(72) Inventor: HAMED MOJAHED, NASHVILLE, TN (US)

(21) Appl. No.: 17/436,106

(22) PCT Filed: Feb. 28, 2020
(86) PCT No.: PCT/EP2020/055345

§ 371 Date: Sep. 3, 2021

Related U.S. Application Data
(60) Provisional application No. 62/814,443, filed on Mar. 6, 2019.

(51) Int. Cl. G16H 40/20; G16H 30/40

(52) U.S. Cl. CPC G16H 40/20; G16H 30/40

(57) ABSTRACT

To enhance user experience and improve patient workflow, data collection and patient imaging protocols are monitored, and real-time notifications are triggered, generated, and provided to end users (e.g. Radiologists, Technologists, service personnel, hospital management, etc.) autonomously, remotely, and in real-time, when a customizable condition is met such as when a scan protocol is finished and reconstructed successfully. The notifications are sent from a scanner host computer or an analysis system and received via email, instant message, etc., and can be pulled into an independent mobile application. Progress indicators are provided, which facilitate informing an attending physician of a predicted scan completion time so that the physician can prepare (e.g. get to the reading room, scanner room, log into mobile reviewing device, etc.) to review the scan results immediately. The user can choose to see aborted, finished, or in-progress scans for any patient, patient group, medical device, or any selected hospital.

### FIELD

The following relates generally to medical imaging systems and the like. However, the described innovation my find application in other imaging systems, other medical systems, etc.

### BACKGROUND

In conditional medical imaging systems, a patient scan is performed, and computer aided diagnosis (CAD) may be applied to autonomously identify suspicious features. The results are transferred to a Picture Archiving and Communication System (PACS) and may be later downloaded to a radiology workstation where a radiologist reviews the images, makes clinical findings, and prepares a radiology report that is uploaded to the PACS and/or sent to one or more clinicians.

In an emergency room setting, magnetic resonance imaging (MRI), computed tomography (CT), or other medical imaging is commonly used to assess and diagnose a patient, where seconds count. For instance, in the United States alone someone suffers from brain stroke every 40 seconds, and a patient who has experienced a stroke may lose up to two million brain cells every minute. If that patient receives an emergency MRI, it is imperative that the physician treating the patient is informed precisely when the MRI scan is complete, and the images have been reconstructed for diagnosing the patient.

However, existing medical imaging scanners are not capable of proactively managing the diagnostic imaging process. To expedite such diagnostic imaging processes, an on-call radiologist may operate on-site at a radiology laboratory, reviewing the diagnostic images in the scanner room as they are generated in a console room at the MRI scanner control computer. In a busy emergency room setting, the radiologist on duty may be moving around between imaging rooms as different trauma patients or other patients are imaged. However, under conventional approaches, the process is inefficient since the radiologist may not be called to a room by an imaging technician to evaluate images until the images become available. Thus, there is a delay as the radiologist assesses the images, makes clinical findings, and so forth. The delay still exists when the acquired images are automatically sent to PACS by the medical devices, simply because there is no efficient way to inform the radiologists to start reviewing the images for diagnosis. It is naïve to assume that the radiologist in charge is always sitting at their viewing station in emergency rooms, reloading the PACS database and waiting for the new patient images to arrive. In some hospitals, the technologist that acquires the medical images calls the radiologist on call or in charge as soon as the patient imaging examination is done, but this method is inefficient because 1) it relies heavily on a middle-man (medical imaging technologist or operator), 2) assumes that the medical imaging technologist knows who to call (e.g., a currently on-duty radiologist), and 3) wastes critical time especially in emergent situations (e.g., two million neurons per minute are lost in a brain stroke and if it is not diagnosed in time, it could increase the risk of permanent brain damage, disability, or even death).

Conventional systems do not provide a way for a radiologist (or end user) to know how their prescribed MR scan protocol (e.g., FLAIR or DWI sequence) is proceeding in time and completion status for a patient that is currently getting an MRI exam (e.g., Brain exam with contrast enhancement which is a collection of one or multiple scan protocols), e.g., whether the MR technologist has completed a critical MR protocol for a patient, whether the images have been loaded into the PACS system, whether the techs omitted an MR protocol by mistake or otherwise deviated from a fixed set of routine protocols that must have been acquired or whether the techs changed parameters they should not have for a particular protocol (because the Radiologists had a certain preference), etc.

Currently, the fastest way to know whether an individual protocol was performed successfully is to check the scanner (or imaging device) database which can cause interruption in the workflow of the scanner. Thus, there is a need for a process in the background to be able to check on extremely important protocol completions and to send a real-time information to the remote users autonomously and without the man-in-the-middle. Assuming a particular necessary protocol was performed and that the images associated with that protocol were sent to PACS database, for a fast access to the data, the radiologist must also wait for data arrival and then refresh the PACS and search for the particular scan protocol, which can potentially waste a lot of the radiologist's time. This is especially true in an emergency department, stroke center, or in a critical care unit where every second matters and there can be multiple imaging devices present or where radiologists have to cover more than one patients. Therefore, radiologists not knowing precisely when the data is available for reading can have devastating consequences for patients.

The following discloses certain improvements.

### SUMMARY

In one disclosed aspect, a medical imaging system that facilitates autonomously providing real-time reconstructed medical image availability notifications comprises: a medical imaging device that acquires image data of a patient; a host computer coupled to the medical imaging device and configured to control one or more imaging protocols executed on the medical imaging device; and a reconstruction processor that reconstructs acquired image data into one or more images. The system further comprises one or more processors configured to: monitor the progress of an imaging protocol that generates at least one image; generate a notification message upon completion of the imaging protocol, the notification message indicating an identity of the imaging protocol and a location at which the at least one image can be reviewed; and transmit the notification message immediately upon completion of the imaging protocol to a communication device of at least one authorized recipient.

In another disclosed aspect, a communication device for autonomously providing real-time notifications of imaging protocol status information, comprises one or more processors configured to: receive a message from a host computer associated with a medical imaging device, the message including a start time and expected duration of each of one or more imaging protocols being monitored by the host computer; for each of the one or more imaging protocols, initiate a progress indicator configured to indicate an amount of time remaining for each monitored imaging protocol; and a graphical user interface on which the progress indicator is presented to a user of the communication device. The one or more processors is fully customizable by the user having the communication device to choose to receive a notification that one or more monitored imaging protocols is complete, and to present via the graphical user interface an instruction to the user to either proceed to or be ready at an exact time in the near future to review images on a mobile device or be at a designated location to review reconstructed images generated via the imaging protocol.

In another disclosed aspect, a non-transitory computer-readable medium has stored thereon instructions for autonomously providing real-time reconstructed medical image availability notifications, the instructions comprising: monitoring progress of an imaging protocol running on a medical imaging device and which generates at least one image; generating and transmitting one or more progress status messages indicative of imagining protocol progress and predicted time of completion; generating a notification message upon completion of the imaging protocol, the notification message indicating an identity of the imaging protocol and a location at which the at least one image can be reviewed; transmitting the notification message immediately upon completion of the imaging protocol to a communication device of at least one authorized recipient.

One advantage resides in providing real-time scan completion notifications to attending radiologists.

Another advantage resides in completely eliminating any time wasted by predicting the availability of images in the future to attending radiologists.

Another advantage resides in improving medical facility workflow by identifying missing protocols, incomplete procedures, or inconsistent parameters used within a protocol.

Another advantage resides in mitigating patient trauma by reducing medical image review delays.

Another advantage resides in providing the results of any analysis performed on the original data (e.g. artificial intelligence algorithms to detect abnormalities) and announcing the preliminary results to attending clinicians in real-time and autonomously, assisting radiologist with diagnosis.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a system that facilitates providing real-time notifications to authorized parties regarding medical imaging scan status, in accordance with one or more features described herein.

FIG. 2 shows a screenshot of an interface running on the Host computer, wherein an email is sent upon fulfilling a condition (e.g. finishing each individual planned protocol).

FIG. 3 shows a screenshot from a user's email account on a desktop computer or the like.

FIG. 4 shows a screen capture from an end user's communication device displaying in real-time the notifications that a hospital 3.0T scanner has sent.

FIG. 5 shows a screen capture from an end user's mobile smart communication device (e.g., an Apple smart watch in this example) displaying the hospital name, medical device type (i.e. MRI), medical device ID (i.e. 3TA), patient type (i.e. Brain Stroke), patient ID, and real time autonomous notifications received on the finished scans (i.e. T1W), aborted scans (i.e. T2W), the scan in progress (i.e. DWI), the countdown clock to predict when the current scan will finish, when the entire exam will finish, and a timer to indicate time elapsed since the onset of the condition that necessitated the scan(s) (i.e. Stroke onset in the example).

### DETAILED DESCRIPTION

The claimed innovation provides immediate information (i.e., immediately after a successful scan protocol is acquired and reconstructed) about the status of each individual scan protocol, by sending an automatic notification message to a selected group of end user recipients.

In clinical MR, for instance, time and connectivity to the actual MR scan progress in real-time are a critical part of the workflow. Notifying end users (i.e. Radiologists, Principal Investigators, etc.,) via email or through a mobile instant messaging (IM) app provides a way of communicating in real-time and facilitates workflow progress by allowing access to the latest examination progress status, to serve patients better and mitigate time waste. Hospital and imaging center management is interested in knowing for consistency purposes whether certain mandatory MR scan protocols are in fact acquired, and if so, successfully or not, as part of an ExamCard (collection of the individual scan protocols), thus enabling them to discover whether ExamCards are modified or deviated from the original form for a given clinical trial. The described systems and methods can be configured such that even a change in imaging slice orientation for a particular scan protocol causes a notification to be sent to a Radiologist in charge. Additionally, service engineers may be interested to know when exactly a long service periodic quality indicator test (PIQT) scan is completed (or interrupted), so they can take necessary additional steps without wasting any time.

The described systems and methods provide “real-time” capability to analyze the patient scan data and notify the clinicians about the results before they had a chance to read the scan data and come up with a diagnosis themselves. For example, a stroke patient is being scanned at an MRI scanner with NotifyMe availability. Upon completion of the scans, artificial intelligence analysis software determines that there is bleeding in one of the arteries. A brief message is provided to the Radiologist, suggesting a possible bleed. The radiologist reads the images and checks the bleeding possibility already suggested through NotifyMe. If true, bleeding was not missed due to human error.

The autonomous NotifyMe does not depend on technologists or other human middlemen, and can send various customizable notifications to any mobile, stationary, or connected devices with Internet access, in real-time for, e.g., the following cases:

availability of clinical data for reading to remote users (e.g. radiologists) in real-time without wasting any moment in emergent situations

announcing the completion of a particular imaging protocol or an entire examination for a patient

displaying a progress bar for completion of a particular imaging protocol or exam

confirmation of sending/receiving a particular clinical image or dataset to PACS or other destinations

predicting when a particular imaging protocol or critical set of protocols are going to be finished in the future (i.e. countdown clock), so the users (e.g. radiologists) can plan ahead to be present at the scanner or reading station for immediate reading of the images.

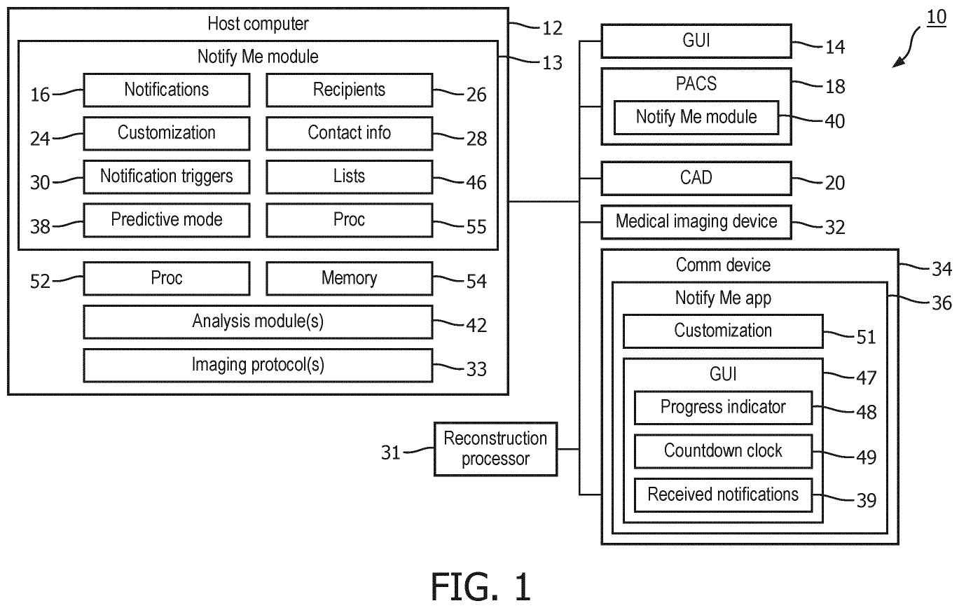

FIG. 1 illustrates a system 10 that facilitates providing real-time notifications to authorized parties regarding medical imaging scan status, in accordance with one or more features described herein. The system 10 may be referred to herein as the “NotifyMe” system. In one embodiment, the system runs on a medical imaging scanner host computer 12, and includes a NotifyMe module 13 comprising a GUI 14 for customizing the types of notifications 16 to be sent, and in certain embodiments provides notifications regarding when a scan is complete, when it is uploaded to the PACS 18, when a CAD analysis 20 is complete, when the imaging technician makes a change to the imaging protocol, or when some other configurable or selectable event(s) occur. The notification message can also include PHI (e.g. the name of the patient) under HIPAA or initials of the patient and/or the scanner number/local name. The GUI 14 also comprises a customization module 24 including selectable options for a physician to indicate, e.g., how often notification updates are to be sent (e.g., every 5 minutes, every 10 minutes, every 30 seconds within 5 minutes of scan completion, etc.), time-of-day restrictions for notifications (e.g., only during the physician's shift, not between certain hours (e.g., 1 a.m. to 6 a.m. or some other predetermined time window, etc.), scanner selection based on name or location or serial number (e.g. only send notifications from the 3.0T scanner at Children's Hospital), generic patient types (e.g. only notify when there is a stroke patient).

In one embodiment, the PACS 18 sends an indication to the host computer 16 to indicate successful upload of scan images. Similarly, the CAD module 20 sends a notification to the host computer indicating that computer-aided diagnosis is complete. In each case, the received indication triggers a notification for the radiologist to be generated and transmitted by the NotifyMe module 13 running on the host computer 12.

The GUI is also used to maintain a set of approved notification recipients 26 along with selectable options for the approved recipient(s). Typically, the approved recipients will include a rotation of on-call radiologists and possibly others such as a rotation of on-call emergency room physicians. For each approved recipient, the system stores appropriate contact information 28 such as email addresses and a cell phone number (e.g., for instant messaging and/or text).

The NotifyMe module 13 running on the scanner computer further includes a real-time events monitor/notification trigger component 30, and a reconstruction processor 31 that reconstructs raw scan data acquired by a medical imaging device 32 into one or more medical images. The real-time events monitor/notification trigger component 30 monitors operation of the medical imaging device 32 (e.g., magnetic resonance imaging (MRI), positron emission tomography (PET), single photon emission tomography (SPECT), computed tomography (CT), ultrasound (UL) or any other suitable medical imaging device) to detect events, which are selected using the GUI, and in real time sends notifications to the approved recipient(s). This feature entails monitoring the imaging protocols 33 executed on the imaging device 32, as well as the image reconstruction process (e.g., to detect when images become available) as well as any CAD analyses. Advantageously, while some operations such as CAD analysis may be performed in the cloud or on a hospital server, the data are sent back to the scanner computer 12, so that the scanner computer is a central point at which all relevant real time image acquisition and processing information accumulates. In another embodiment, notifications are sent directly from a connected CAD system (for example if the analysis software is for detecting lesions, and that software is residing on a separate server, then the trigger for the notification can directly come from that server.) This feature allows the NotifyMe module 13 running on the scanner computer to instantly identify events that trigger notifications. Existing scanner computers are typically already connected with the Internet (possibly via a firewall) so that the NotifyMe module running on the scanner computer can send notification emails and/or instant messaging (IM) messages to the approved recipients.

The approved recipient receives the notification via the Internet (or possibly via a wireless hospital network) at a mobile communication device 34, such as a cell phone or smart device (smartphone, smartwatch, or the like) or “Internet of Things” (IOT) device or other mobile communication device (e.g., a tablet, personal computer, etc.) worn or carried by the approved recipient. The notification can be a standard email or IM message, in which case an email or IM app can provide the notification user interface. Additionally or alternatively, a dedicated NotifyMe app 36 can be loaded onto the mobile communication device, in which case the notification can be sent via a proprietary protocol. Use of a dedicated NotifyMe app can provide greater flexibility and Patient Health Information and can enable additional features as mentioned below.

In one embodiment, rather than providing a notification at the time an event occurs in real time, the NotifyMe module running on the scanner host computer may operate in a predictive mode 38. For example, the NotifyMe module can detect the start of a scanning sequence, predict when the scanning sequence will finish (e.g., in 20 minutes) based on the known scan duration, and push a notification 39 to the NotifyMe app 36. At the NotifyMe app, a 20-minute timer is started so that the approved recipient can know when he or she needs to be at the reading room, or the scanner computer 12 in the console room to immediately review the results when they become available. The system can also predict the precise time of the availability of data so the end users can view the results without any life-costing delay. The notifications can be sent to end users immediately after an analysis software provides results on the data, to assist clinicians with decision making.

This feature reduces the amount of time a radiologist must spend stopping by one or more console rooms to check whether the scan is complete, thereby freeing up time for the radiologist to check on patients and generally be more efficient. This is especially useful in emergent MR where the radiologists prefer to walk into the scanner room instead of relying on the PACS system to remotely read the time sensitive imaging data.

Another feature the NotifyMe app provides is an enhanced ability for the approved recipient to communicate back to the imaging technician in real time. This can be especially valuable in situations where the notification relates to a change in the scan parameters or protocol, such as when patient moves during a scan and the scan must be restarted, or when the imaging technician determines that the scan requires additional time or an additional procedure. Additionally, if the radiologist does not approve such changes, he or she can immediately message to the technician to countermand the change. The physician can also use the NotifyMe app 36 to send instructions to the imaging technician, such as instructions to repeat a scan, perform an additional scan, etc.

The NotifyMe module 13 runs on the scanner computer 12 and monitors the scanning in real time in order to monitor any image analyses that are performed manually e.g. using an image reviewing GUI) or by CAD, even if performed at a remote/cloud server. In one embodiment, a NotifyMe module 40, which is configured to perform any or all of the functions of the NotifyMe module 13, may run on the PACS 18 and is configured to detect receipt of the scan examination at the PACS. This feature can facilitate performing a data integrity check and true real time notification regarding when the scan results become available at the PACS 18.

The system further comprises one or more analysis modules 42, which analyze reconstructed scan images to detect abnormalities. Notification messages generated by the NotifyMe module 33 can include an indication regarding whether the scan results are normal or abnormal. For instance, if a physician orders an MRI of a patient's brain, the analysis module determines whether one or more lesions are present in the brain and transmits to the NotifyMe module an indication that the scan is abnormal. The NotifyMe module converts the abnormal indication to a message format (text, IM, email, etc.) and includes the abnormal indication in the notification message. This feature facilitates providing real-time abnormality notifications to the physician. The analysis module 42 can reside on the host scanner computer 12 or may reside on a remote server or in the “cloud.”

According to one embodiment, an attending physician can access the NotifyMe application on the host scanner computer 12 or the NotifyMe application on their communication device to configure which notifications 16 the physician would like to receive, how frequently the physician would like to receive each notification, etc. For instance, the physician (e.g., a radiologist or the like) can select to have a scan progress notification sent to his communication device (e.g., smartphone, smartwatch, tablet, laptop, or the like) at predetermined intervals (e.g., every 10 minutes, 5 minutes, 30 seconds, etc.) for a given patient and/or a given scan procedure. The notification message sent to the physician's communication device can comprise a message (e.g., email, IM, text, MMS, etc.) indicating a status of the scan procedure (e.g., complete or ready for review, time remaining until complete, delayed, etc.) in real-time. Additionally or alternatively, each scan completion can trigger a notification to one or more authorized recipients 26.

In one embodiment, notification frequency is increased as scan completion approaches. For example, a notification schedule for a 20-minute scan can comprise sending a progress notification every 5 minutes until the scan is within 3 minutes of completion, then every 30 seconds until the scan is within 1 minute of completion, then every 5 seconds for the last minute until the scan is complete. This feature facilitates conveying a sense of urgency to the physician to be present to read the scan results immediately upon availability. In a related embodiment, the user (physician or the like) can assign different tones on the receiving communication devices to different scans or patients so that the user can distinguish between different scan results becoming imminently available without looking at the communication device. In one example, the user can assign higher-pitched tones to higher priority patients on the cellphone application.

The physician is also permitted to generate one or more lists 46 comprising patients, imaging devices, etc., which may be prioritized or ranked, via the NotifyMe module. For instance, the physician can rank patients in order of priority or injury severity.

In another embodiment, the NotifyMe app on the physician's communication device includes a GUI 47 comprising a progress status indicator 48 such as a countdown clock 49, timer, status bar, or the like, that indicates when the scan results will be available for review. For instance, the physician can be presented with a status bar that indicates the scan is 10%, 50%, 100% complete, or some other percentage. In another embedment, the NotifyMe app includes a countdown clock or status bar that indicates when the scan results will be available for review, and the physician can monitor the countdown in order to be present in the correct reading room to review the scan results the moment they are available or several minutes beforehand.

The progress indicator 48 is also customizable by the physician via the NotifyMe module 13 on the host computer and/or via the NotifyMe app 36 on the communication device 34. For instance, the physician can choose whether the progress indicator is depicted as a countdown timer, clock, or status bar, or the like. Additionally, the physician can select one or more times to receive notifications regarding the pending completion of each imaging scan (e.g., t-minus 5 minutes, t-minus 30 seconds, etc.). The foregoing features facilitate reducing an amount of time between the end of a scan procedure and radiologist review of the scan results, which in turn improves patient recovery (e.g., by mitigating brain cell death in the case of the stroke patient, etc.), improves patient outcome, prevents delay-related injuries, and reduces irreversible damage caused by a time-sensitive disease like brain stroke.

By notifying the physician in advance of the predicted availability of the scan results, physician efficiency and likelihood of patient recovery are improved. For instance, a physician who is monitoring 10 patients and awaiting scan results for each patient can be alerted to be present at a given scanner or reading room in advance of or at the moment of scan result availability for each patient. Additionally, the physician can use the NotifyMe app on the physician's communication device to prioritize patient scan result availability notifications. For instance, the physician can rank patients in order of patient conditions or maladies, such as prioritizing a stroke victim MRI scan over a tennis elbow MRI scan, so that in the event that both patient's scan results become available concurrently, the physician is not reviewing the less critical scan results to the detriment of the more critical patient.

Additional features provided by the NotifyMe architecture can include notifications to the ordering physician or radiologist, or imaging facility management, regarding deviations from an ordered scan. For instance, a tech may restart a scan or omit a portion of an ordered scan, and such an event triggers a notification message. This feature facilitates detecting “drift” in real time. Conventional approaches typically involve retrospective analysis of, e.g., a year's worth of scan procedures in order to identify “drift” (deviations from scan procedures). Deviation from a particular set of parameters for a given scan protocol can be detected in real-time. For example, if an MR Technologist is told to run a particular scan protocol without any modifications, but they accidentally modify the echo time, NotifyMe can send an alert to the management, notifying them about a potential drift in the scan protocol (current gold standard is to detect the drift after the fact, for a prior period of time). By providing real-time drift detection, facility management, service technicians, and even physicians can have access to more timely information that facilitates revising scan procedures and workflow.

For instance, while technicians start with a standard imaging protocol, over time due to modifications made by various technicians, the protocol stored in the host computer changes. The NotifyMe module 13 can be configured to notify a technician of the protocol drift (e.g., variations from the standard protocol) by comparing a current imaging protocol to a standard or base protocol (e.g., an original or standard version of the protocol). In one embodiment, the NotifyMe module can generate and transmit a notification if the technician makes certain modifications to, e.g., an MRI protocol. In another embodiment, a notification is generated and transmitted if the modification(s), respective to a base scan protocol, exceeds a predetermined threshold (e.g., 5% variation, 10% variation, or some other predetermined amount). This feature ensures that a notification is provided if successive adjustments to the protocol over time reach the point where the presently-run protocol has drifted more than the predetermined threshold amount from the original (base) scan protocol.

The communication device 34 provides real-time notifications of imaging protocol status information and comprises one or more processors (not shown) configured to receive a message from the host computer 12 associated with a medical imaging device, wherein the message includes a start time and expected duration of each of one or more imaging protocols being monitored by the host computer. The one or more processors may be similar to the one or more processors 52 (described below) with regard to the host computer 12. It will be understood that the communication device also comprises computer-readable media or memory(ies) (not shown) such as may be employed in a communication device such as a smartphone, smart watch, internet-of things-device, or the like).

For each of the one or more imaging protocols, the one or more processors initiate a progress indicator configured to indicate an amount of time remaining for each monitored imaging protocol. The communication device also comprises a graphical user interface 47 on which the progress indicator is presented to a user of the communication device. The one or more processors is further configured to receive a notification that one or more monitored imaging protocols is complete, and present via the graphical user interface an instruction to the user to proceed to a designated location to review reconstructed images generated via the imaging protocol. The communication device is further configured to receive one or more update messages that update a predicted end time of the one or more imaging protocols and update the progress indicator as a function of the updated end time. In one embodiment, the NotifyMe application running on the communication device comprises a customization component 51 that is similar or identical to the customization component 24 described with regard to the host computer 12. In this regard, the NotifyMe application 36 can provide any or all of the functionality, via the communication device (e.g., customization, list generation, etc.) provided by the NotifyMe module 13 from the user standpoint. For instance, the end user can set the how often to receive alerts, choose the scanners from which the alerts are received, select a specific patient (e.g., under HIPAA), select generic patient type (e.g. brain stroke patients only), days of the week to receive the notifications, time of the day to receive the notifications, etc.

The NotifyMe application (and/or processors on which the application runs) is configured to receive a selectable user input regarding, for each patient undergoing an imaging protocol, at least one of: a desired date or time of receipt of one or more progress status notifications from the host computer prior to imaging protocol completion; a desired frequency of transmission of progress status notification messages; and a desired period of non-transmission of progress status notification messages.

In another embodiment, the NotifyMe application further configured to receive user input regarding an identity of at least one communication device of an authorized recipient to which notifications and progress status messages may be transmitted, and to generate a list of one or more authorized recipients. The user input can include a patient ID number for a patient undergoing an imaging protocol, wherein the list of authorized recipients comprises one or more physicians responsible for the patient, and wherein NotifyMe application configured to transmit the list of authorized recipients to the host computer for distribution of messages and notifications.

In one embodiment, the notification is received from the host computer after a computer-aided diagnostic (CAD) component has analyzed acquired scan data and provides diagnostic information. In another embodiment, the notification is received from the host computer after a post-processing component has analyzed reconstructed image data and provided an indication to the host computer regarding whether the reconstructed image data comprises an abnormality. The notification can also include an indication regarding whether the at least one image comprises an abnormality.

In one embodiment, the NotifyMe module 13 stores and executes a Perl script that includes and calls other Perl packages, and in real-time constructs a notification message (e.g., email, instant message, text, etc.) if certain criteria, which are customizable, are met (e.g. successful completion of a particular scan protocol acquisition, or full availability of all reconstructed images for the end users, etc.), and then establishes a connection (email, IM, text) and sends the notification message immediately. The Perl packages are individual files and folders that can be copied to the main scanner host computer 12.

The host computer 16 also comprises one or more processors 52 and one or more memories 54 (computer-readable media). The processor 52 executes, and the memory 54 stores, one or more computer-executable modules for performing the various functions, methods, etc., described herein. “Module,” as used herein, denotes a computer-executable algorithm, routine, application, program, or the like, and/or a processor that executes said computer-executable algorithm, routine, application, program, or the like.

The memory 54 may be a computer-readable medium on which a control program is stored, such as a disk, hard drive, or the like. Common forms of computer-readable media include, for example, floppy disks, flexible disks, hard disks, magnetic tape, or any other magnetic storage medium, CD-ROM, DVD, Blu-Ray or any other optical medium, RAM, ROM, PROM, EPROM, FLASH-EPROM, variants thereof, other memory chip or cartridge, or any other tangible medium from which the processor 52 can read and execute. In this context, the described systems may be implemented on or as one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphics processing unit (GPU), or PAL, or the like.

In another embodiment, the NotifyMe module comprises one or more dedicated processors 55 that perform the various actions, functions, tasks, etc., described herein. For instance, the processor(s) 55 can parse a system-generated log in the background in real time and take action (e.g., send notifications, predictions, etc.) to authorized users.

According to one example, the following Perl packages can be used inside the NotifyMe.pl file, e.g., to establish an email connection:
i.Email::Send (http://search.cpan.org/dist/Email-Send/lib/Email/Send.pm)
ii.Email::Send::Gmail (http://search.cpan.org/˜lbrocard/Email-Send-Gmail-0.33/lib/Email/Send/Gmail.pm)
iii.Email::Simple::Creator (http://search.cpan.org/˜rjbs/Email-Simple-2.214/lib/Email/Simple/Creator.pm)
iv.Other Perl packages used inside the NotifyMe.pl file may include “Cwd”, “File::Copy”, “strict”, and “warnings”.

In another embodiment, there are dependent Perl packages that can be automatically pulled from the CPAN website (http://search.cpan.org/), in the event that any are missing. Perl is installed on scanners running NotifyMe by default, but in the event that any Perl packages need to be added for the NotifyMe.pl script to function properly on a particular scanner, this feature is advantageous.

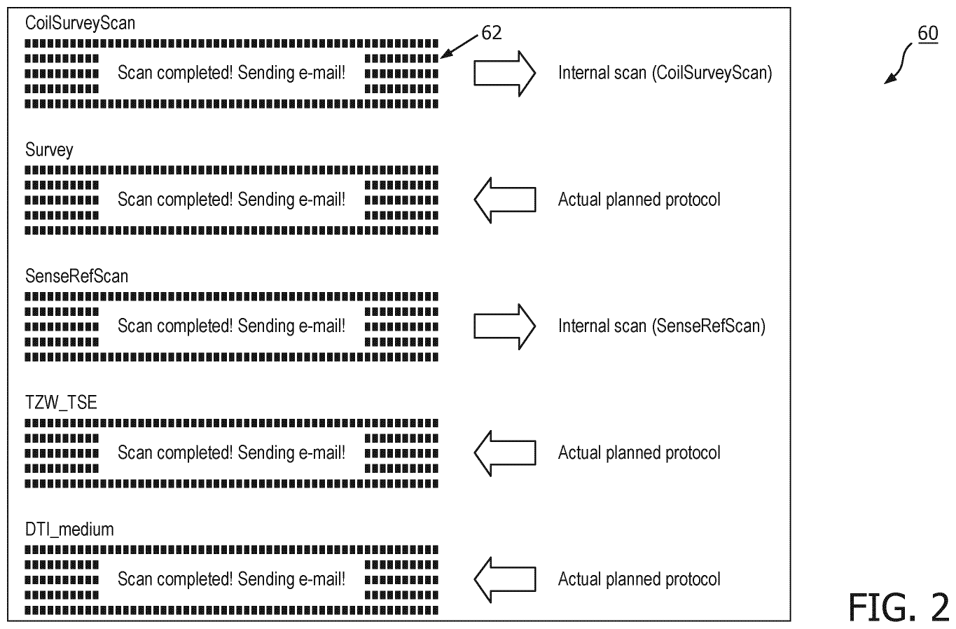

FIGS. 2-4 below show an example of the described NotifyMe tool implemented on the Host scanner computer 12 and the resulting remote notifications on the end user's desktop or mobile device.

FIG. 2 shows a screenshot 60 of an interface running on the host computer, wherein an email 62 is sent upon fulfilling a condition (e.g. finishing each individual planned protocol). In the illustrated example, three patient scan protocols are planned and completed successfully, but in actuality five protocols are executed because there are two additional internal scans (CoilSurveyScan and SenseRefScan) that are acquired in the background. In this example, a user (e.g., a radiologist) has opted or selected to include the internal scans and receive all five email notifications. However, the herein-described NotifyMe system permits the user to configure settings to receive all or fewer than all scan completion notifications.

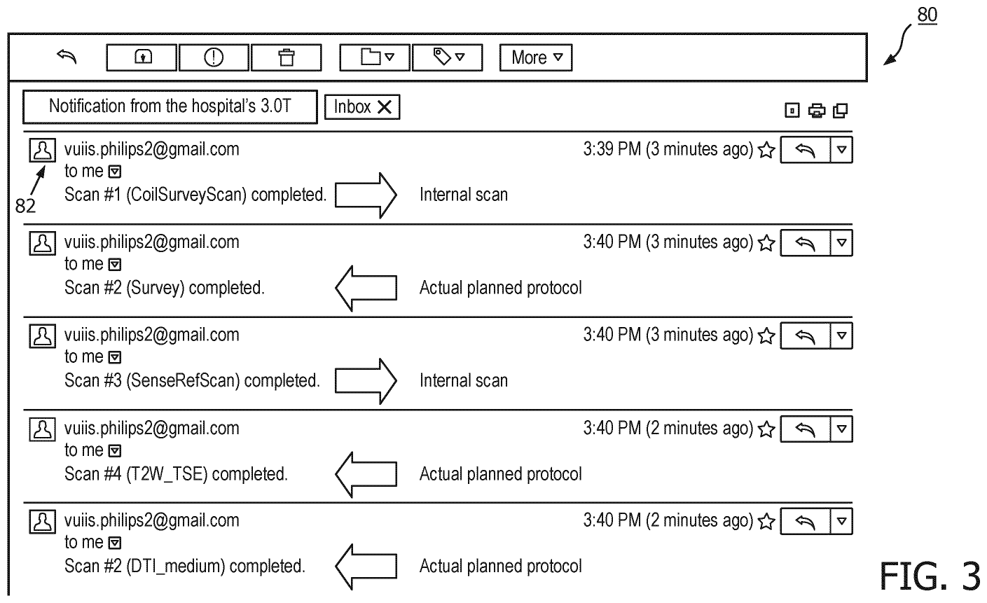

FIG. 3 shows a screenshot 80 from an authorized user's email account on a desktop computer or the like. In the illustrated example, resulting notification emails 82 are received from a hospital's 3.0T scanner via messages (e.g., email, IM, text, etc.), indicating true scan numbers and protocol names. Both internal scans and planned protocols are displayed. HIPAA privacy rules regarding any patient health information are strictly followed in independent mobile applications.

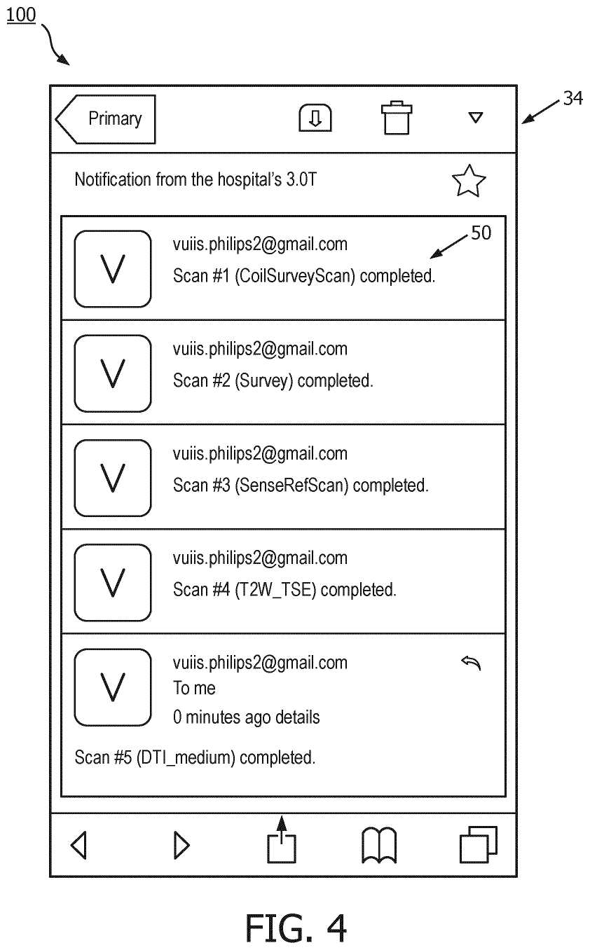

FIG. 4 shows a screen capture 100 from an end user's communication device 34 displaying in real-time the notifications 16 that a hospital 3.0T scanner has sent, which appear on the communication device as received notifications 39, in this example upon completion of individual imaging protocols.

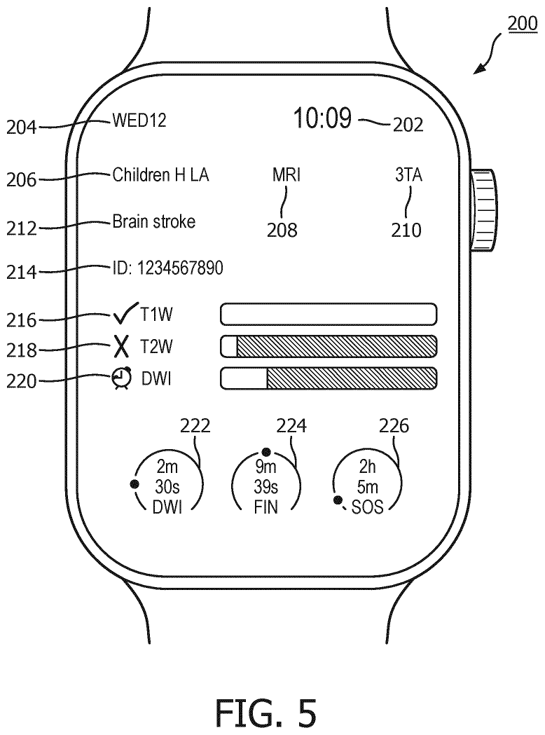

FIG. 5 illustrates an example of a NotifyMe interface 200 such as can be displayed on a user's communication device (e.g., a smartwatch, smartphone, etc.) in accordance with one or more aspects described herein. The interface 200 shows current date 202, current time 204, hospital name 206, scanning device type 208 (MRI in the illustrated example), and scanner device name or ID 210 (3TA in the illustrated example). The interface additionally shows a patient type 212 (a brain stroke patient in the illustrated example), and patient ID information 214. Several scan status indicators are provided, including but not limited to a scan complete indicator 216 (T1W in the illustrated example), scan aborted indicator 218 (T2W in the illustrated example), and a scan-in-progress indicator 220 (DWI in the illustrated example). A plurality of progress indicators is also shown, including a current scan countdown indicator 222 (the DWI scan in the illustrated example), a countdown to finish of the entire examination 224 (FIN in the illustrated example), and a time elapsed indicator 226 that indicates time elapsed since the onset of the condition that necessitated the scan(s) (stroke in the illustrated example). It will be appreciated that the described example need not limited to the particular a arrangement of indicators and/or information on the interface display, and that more or less information, indicators, etc., may be presented via the interface 200 in accordance with various embodiments.

The invention has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A medical imaging system that facilitates autonomously providing real-time reconstructed medical image availability notifications, comprising:
a medical imaging device that acquires image data of a patient;
a host computer coupled to the medical imaging device and configured to control one or more imaging protocols executed on the medical imaging device;
a reconstruction processor that reconstructs acquired image data into one or more images; and
one or more processors configured to:
monitor the progress of an imaging protocol that generates at least one image;
generate a notification message upon completion of the imaging protocol, the notification message indicating an identity of the imaging protocol and a location at which the at least one image can be reviewed or stored; and
transmit the notification message immediately upon completion of the imaging protocol to a communication device of at least one authorized recipient.

2. The system according to claim 1, further comprising a customization module configured to receive during setup user input regarding an identity of at least one communication device of an authorized recipient to which the notification is transmitted, and to populate a list of authorized recipients to which the notification is transmitted.

3. The system according to claim 2, wherein the user input includes the list of authorized recipients comprising one or more physicians responsible for the patient, and wherein a patient ID number for a patient undergoing the imaging protocol is read by the processing unit of the NotifyMe module on the host.

4. The system according to claim 1, wherein the one or more processors is further configured to predict when the imaging protocol will be completed based on a known start time and a known duration of the imaging protocol, and to generate and transmit one or more real-time progress status notification messages to the communication device of the at least one authorized recipient.

5. The system according to claim 4, further comprising a customization component configured to receive a selectable user input during regarding, for each patient undergoing an imaging protocol, at least one of:
a desired time of receipt of the one or more progress status notifications prior to imaging protocol completion;
a desired frequency of transmission of progress status notification messages; and
a desired period of non-transmission of progress status notification messages.

6. The system according to claim 1, wherein the one or more processors is further configured to initiate and periodically update a progress indicator on the communication device of the at least one authorized recipient, wherein the progress indicator indicates an expected time of completion of the at least one imaging protocol.

7. The system according to claim 1, further comprising a computer-aided diagnostic (CAD) component that analyzes acquired scan data and provides real-time diagnostic information in order to assist clinicians with their diagnosis and decision making.

8. The system according to claim 1, further comprising an analysis module that analyzes reconstructed image data and provides a real-time indication to the one or more processors regarding whether the reconstructed image data comprises an abnormality.

9. The system according to claim 8, wherein the notification message includes an indication that the imaging protocol is complete and an indication regarding whether the at least one image comprises an abnormality, in real-time.

10. The system according to claim 1, wherein the medical imaging device is one of a magnetic resonance imaging (MRI) device, a computed tomography (CT) scanner, a positron emission tomography (PET) scanner, a single photon emission computed tomography (SPECT) scanner, and an ultrasound (UL) imaging device.

11. A communication device for autonomously providing real-time notifications of imaging protocol status information, comprising:
one or more processors configured to:
receive a message from a host computer associated with a medical imaging device, the message including a start time and expected duration of each of one or more imaging protocols being monitored by the host computer;
for each of the one or more imaging protocols, initiate a progress indicator configured to indicate an amount of time remaining for each monitored imaging protocol; and
a graphical user interface on which the progress indicator and a countdown clock are presented to a user of the communication device;
wherein the one or more processors is further configured to receive a notification that one or more monitored imaging protocols is complete or about to be completed in a predicted amount of time as indicated by the countdown clock, and to present via the graphical user interface an instruction to the user to proceed to review reconstructed images available at a particular location.

12. The communication device according to claim 11, wherein the one or more processors is further configured to receive one or more update messages that update a predicted end time of the one or more imaging protocols, and update the progress indicator as a function of the updated end time.

13. The communication device according to claim 11, further comprising a customization component that is configured to receive a selectable user input regarding, for each patient undergoing an imaging protocol, at least one of:
a desired time prior to imaging protocol completion for the receipt of one or more progress status notifications from the host computer;
a desired weekday and time for the receipt of the notifications;
a desired scanner or set of dedicated scanners in a particular hospital section;
a desired specific patient or generic patient types to receive notifications about;
a desired frequency of receipt of progress status notification messages; and
a desired period of non-receipt of progress status notification messages.

14. The communication device according to claim 13, wherein the customization module is further configured to receive during setup user input regarding an identity of at least one communication device of an authorized recipient to which notifications and progress status messages may be transmitted, and to generate a list of one or more authorized recipients.

15. The communication device according to claim 14, wherein the user input includes a patient ID number for a patient undergoing an imaging protocol, wherein the list of authorized recipients comprises one or more physicians responsible for the patient, and wherein the one or more processors is configured to transmit the list of authorized recipients to the host computer for distribution of messages and notifications.

16. The communication device according to claim 11, wherein the notification is received from the host computer after a computer-aided diagnostic (CAD) component has analyzed acquired scan data and provides diagnostic information in real-time, prior to the clinicians having a chance to review the data.

17. The communication device according to 11, wherein the notification is received from the host computer after a post-processing component has analyzed reconstructed image data and provided an indication to the host computer regarding whether the reconstructed image data comprises an abnormality.

18. The communication device according to claim 17, wherein the notification includes an indication regarding whether the at least one image comprises an abnormality.

19. The communication device according to claim 11, wherein the monitored medical imaging protocol is one of a magnetic resonance imaging (MRI) protocol, a computed tomography (CT) protocol, a positron emission tomography (PET) protocol, a single photon emission computed tomography (SPECT) protocol, and an ultrasound (UL) imaging protocol.

20. (canceled)

21. (canceled)

22. A non-transitory computer-readable medium having stored thereon instructions for autonomously providing real-time reconstructed medical image availability notifications, the instructions comprising:
monitoring progress of an imaging protocol running on a medical imaging device and which generates at least one image;
generating and transmitting one or more progress status messages indicative of imagining protocol progress and predicted time of completion;
generating a notification message upon completion of the imaging protocol, the notification message indicating an identity of the imaging protocol and a location at which the at least one image can be reviewed;
transmitting the notification message immediately upon completion of the imaging protocol to a communication device at least one authorized recipient.
